(19) United States
(12) Patent Application Publication
(10) Pub. No.: US 2012/0020891 A1
(43) Pub. Date: Jan. 26, 2012

(54) METABOLITES FOR ORAL HEALTH AND USES THEREOF

(75) Inventors: Virginia M. Barnes, Ringoes, NJ (US); Harsh M. Trivedi, Somerset, NJ (US); Tao Xu, Newton, NJ (US)

(73) Assignee: Colgate-Palmolive Company, New York, NY (US)

(21) Appl. No.: 13/257,988

(22) PCT Filed: Apr. 1, 2009
(86) PCT No.: PCT/US2009/039184

§ 371 Date: Sep. 21, 2011

(51) Int. Cl. A61K 49/00; C07C 43/295; H01J 49/28; C12Q 1/02

(52) U.S. Cl. 424/9.7; 435/29; 568/638; 250/282

(57) ABSTRACT

The present invention provides various methods of using metabolite profiles correlated with periodontal disease or a health oral status for the diagnosis of periodontal disease, identification of responders and, or non-responders to therapeutic agents for periodontal disease, a method to test the efficacy of test compounds to prevent periodontal disease. The present invention also provides for a dentifrice composition containing an effective amount of a metabolite therapeutic agent which brings about a greater change in metabolite levels compared to a control dentifrice composition.

### FIELD OF THE INVENTION

The present invention relates to the differential expression profiles of metabolites in periodontal diseases and methods of diagnosing periodontal diseases based upon these differential expression profiles. The present invention further relates to methods of predicting and/or evaluating the efficacy of therapeutic agents for periodontal diseases based upon the differential expression profiles.

### BACKGROUND OF THE INVENTION

Periodontal diseases are among the most common infectious diseases in humans (Pihlstrom et al., 2005). Aside from affecting oral tissues, periodontal diseases have also been associated with various systemic diseases (Seymour et al., 2007). Gingivitis, the mild form of the diseases, is characterized by host tissue inflammation and bacterial plaque accumulation around the gingival margin. Treatment of gingivitis by improved oral hygiene practices can significantly reverse the disease condition. However, left untreated, gingivitis can lead to the more serious and irreversible periodontitis, which involves progressive loss of the alveolar bone around the teeth, and if left untreated, can lead to the loosening and subsequent loss of teeth.

The majority of the complex interactions between host tissues and bacteria in periodontal diseases occur at the junctional and crevicular epithelia. Many substances released by bacteria, such as endotoxins, proteases, lipases and sialidases, have been demonstrated to play significant roles in host tissue damage (Smalley et al., 1994). However, increasing evidence suggests that the diseases are also mediated by the host's inflammatory responses to bacteria (Van Dyke and Serhan, 2003). Under activation of various chemical signals, the host tissues orchestrate a range of complex responses to combat bacteria. Polymorphonuclear leucocytes produce increasing levels of reactive oxygen species (ROS) and proteolytic enzymes. Hyperactivity of this response can inadvertently contribute to the host tissue damage.

At the interface of the epithelia and bacterial plaque is plasma-derived gingival crevicular fluid (GCF). Because GCF can be collected non-invasively and is site specific, it is an ideal matrix to study host-bacteria interactions (Embery and Waddington, 1994). Using a variety of targeted biochemical analyses, many potential GCF markers for periodontal disease have been proposed, including: host and bacterial enzymes, endotoxins, nucleic acids, proteins, carbohydrates and lipids, degradation products from collagens and bones, immunoglobulins, cytokines and hormones (Embery and Waddington, 1994; Prapulla et al., 2007; Karthikeyan and Pradeep, 2007; Akalin eta, 2007; Pradeep et al, 2007). However, despite the wealth of information published, the broad extent of host-bacteria interactions and the mechanistic details of disease progression on cellular biochemical metabolism still lack clarity.

### SUMMARY OF THE INVENTION

The present invention provides for a method of diagnosing oral health in a subject in which a gingival crevicular fluid sample is collected from the subject and a level of one or more metabolites in the gingival crevicular fluid sample is detected. The subject is diagnosed as having periodontal disease or healthy oral status based on the level of the detected metabolite.

The present invention also discloses a method for diagnosing oral health in a subject in which a gingival crevicular fluid sample is collected from the subject and a level of one or more metabolites in the gingival crevicular fluid sample is detected. The level of detected metabolite in the gingival crevicular fluid sample is compared to a metabolite reference level to thereby generate a differential level. The metabolite reference level corresponds to one or more of the following: periodontal reference level or healthy reference level. In one embodiment, the differential level between the detected metabolite and the periodontal reference level correlates with periodontal disease. In another embodiment, the differential level between the detected metabolite and the healthy reference level correlates with healthy oral status.

In such embodiments, the detected metabolite may be chosen from: a compound generated by amino acid metabolism, a compound generated in urea cycle; compound generated in glutathion conversion; a compound generated in lipid metabolism; a compound generated in carbohydrate metabolism; a compound generated by nucleic acid metabolism; vitamins; and co-factors.

The invention also provides a method for predicting a subject's response, e.g., responder or non-responder, to using a therapeutic agent for periodontal disease while following a standard care protocol. A metabolite profile of a gingival crevicular fluid sample collected from a test subject is generated, wherein the metabolite profile includes the metabolite identity and metabolite level. The metabolite profile of the test subject is compared to a reference metabolite profile. The reference metabolite profile may includes one or more of: a reference responder metabolite profile and a reference non-responder metabolite profile. The results of the comparison can be used to identify the test subject as responder or non-responder to therapeutic agents. The reference metabolite profile can be obtained from subjects who benefited from the standard therapeutic agent, with regression of periodontal disease, or prevention of periodontal disease. This method could be used to determine whether a test subject is a suitable subject to participate in a clinical trial of test therapeutic agent(s).

The invention also pertains to a method for predicting a test subject's response, e.g., responder or non-responder, to development of periodontal disease while following a standard non-care protocol. A metabolite profile of the gingival crevicular fluid sample collected from the subject is generated, wherein the metabolite profile includes the metabolite identity and metabolite level. The metabolite profile of the test subject is compared to a reference metabolite profile, wherein the reference metabolite profile is generated from a reference responder subject and reference non-responder subject. The reference metabolite profile includes the reference metabolite identity and reference metabolite level. The results of the comparison can be used to identify the test subject as responder or non-responder to periodontal disease development.

The present invention further provides for an oral care test kit which may provide the user an indication of the user's oral health status. The kit may include one or more gingivitis crevicular fluid collection strips and a diagnosis of the subject's oral health status. The gingivitis crevicular tluid collection strips may be used for collecting a gingival crevicular fluid sample and for recovery of metabolites contained in the gingival crevicular fluid sample. The diagnosis of a subject's oral health may be based on the methods of this invention.

The present invention further provides for a dentifrice composition. The composition may include an effective amount of a metabolite therapeutic agent. The therapeutic agent effects a change in metabolite levels over a time period of at least one month wherein the change metabolite level is greater than a corresponding change in metabolite reference levels affected by a control dentifrice composition.

In accordance with yet another aspect, the present invention provides for a metabolite indicating dentifrice and its method of use wherein the dentifrice includes a metabolite indicating composition which presents a user discernable indicator upon exposure to a metabolite and a metabolite level associated with periodontal disease. In one embodiment, the user discernable indicator corresponds to a change in color of the dentifrice.

### DETAILED DESCRIPTION OF THE INVENTION

As used throughout, ranges are used as shorthand for describing each and every value that is within the range. Any value within the range can be selected as the terminus of the range. In addition, all references cited herein are hereby incorporated by reference in their entireties.

### DEFINITIONS

As used herein, the term “differential level” of a metabolite may include any increased or decreased level. In one embodiment, differential level means a level that is increased by: at least 5%; by at least 10%; by at least 20%; by at least 30%; by at least 40%; by at least 50%; by at least 60%; by at least 70%; by at least 80%; by at least 90%; by at least 100%; by at least 110%; by at least 120%; by at least 130%; by at least 140%; by at least 150%; or more. In another embodiment, differential level means a level that is decreased by: at least 5%; by at least 10%; by at least 20%; by at least 30%; by at least 40%; by at least 50%; by at least 60%; by at least 70%; by at least 80%; by at least 90%; by at least 100% (i.e., the metabolite is absent). A metabolite is expressed at a differential level that is statistically significant (i.e., a p-value less than 0.05 and/or a q-value of less than 0.10 as determined using, either Student T-test, Welch's T-test or Wilcoxon's rank-sum Test).

As used herein “gingival crevicular fluid” means fluid found around the gingival including the gum; the mucous membrane, with supporting fibrous tissue, covering the tooth-bearing border of the jaw.

As used herein “gingivitis” means an irritation of the gums caused by bacterial plaque that accumulates in the small gaps between the gums and the teeth and by calculus that forms on the teeth.

As used herein “healthy oral status” means the absence of gingivitis and/or periodontal disease.

As used herein, the term “level” of one or more metabolites means the absolute or relative amount or concentration of the metabolite in the sample.

As used herein, the term “metabolite” means any substance produced by metabolism or necessary for or taking part in a particular metabolic process. The term does not include large macromolecules, such as large proteins (e.g., proteins with molecular weights over 2,000, 3,000, 4,000, 5,000, 6,000, 7,000, 8,000, 9,000, or 10,000); large nucleic acids (e.g., nucleic acids with molecular weights of over 2,000, 3,000, 4,000, 5,000, 6,000, 7,000, 8,000, 9,000, or 10,000); or large polysaccharides (e.g., polysaccharides with a molecular weights of over 2,000, 3,000, 4,000, 5,000, 6,000, 7,000, 8,000, 9,000, or 10,000). The term metabolite includes signaling molecules and intermediates in the chemical reactions that transform energy derived from food into usable forms including, but not limited to: sugars, fatty acids, amino acids, nucleotides, antioxidants, vitamins, co-factors, lipids, intermediates formed during cellular processes, and other small molecules.

As used herein “periodontal disease” means an inflammation of the periodontium including the gingival, or gum tissue; the cementum, or outer layer of the roots of teeth; the alveolar bone, or the bony sockets into which the teeth are anchored; and the periodontal ligaments which are the connective tissue fibers that run between the cementum and the alveolar bone and includes gingivitis.

As used herein, the term “reference level” of a metabolite means a level of the metabolite that is indicative of a particular disease state, oral status, phenotype, or lack thereof, as well as combinations of disease states, phenotypes, or lack thereof. In one embodiment, a periodontal reference level or a metabolite means a level of the metabolite that is indicative of a positive diagnosis of periodontal disease in a subject. In another embodiment, a “healthy reference level” of a metabolite means a level of a metabolite that is indicative of a positive diagnosis of a healthy oral status in a subject.

In one embodiment, a “reference level” of a metabolite may be an one or more of the following: absolute or relative amount or concentration of the metabolite; a presence or absence of the metabolite; a range of amount or concentration of the metabolite; a minimum and/or maximum amount or concentration of the metabolite; a mean amount or concentration of the metabolite; and/or a median amount or concentration of the metabolite. In another embodiment, “reference levels” for combinations of metabolites may also be ratios of absolute or relative amounts or concentrations of two or more metabolites with respect to each other. Appropriate positive and negative reference levels of metabolites for a particular disease state, phenotype, or lack thereof may be determined by measuring levels of desired metabolites in one or more appropriate subjects, and such reference levels may be tailored to specific populations of subjects (e.g., a reference level may be age-matched so that comparisons may be made between metabolite levels in samples from subjects of a certain age and reference levels for a particular disease state, phenotype, or lack thereof in a certain age group). In another embodiment, the reference levels may be tailored to specific techniques that are used to measure levels of metabolites in biological samples (e.g., LC-MS, GC-MS, etc.), where the levels of metabolites may differ based on the specific technique that is used.

In another such embodiment, “a reference metabolite” may include at least one compound chosen from: a compound generated by amino acid metabolism, a compound generated in urea cycle; a compound generated in glutathion conversion; a compound generated in lipid metabolism; a compound generated in carbohydrate metabolism; a compound generated by nucleic acid metabolism; vitamins; and co-factors. In another embodiment, the “reference metabolite” may include one or more of compounds listed in Tables 1, 2, 3, 4 and 5. In still another embodiment, the “reference metabolites” may include one or more of compounds inosine, hypoxanthine, xanthine, guanosine, guanine, leucine, isoleucine, lysie, methionine, phenyllalanine, proline, serine, threonine, tryptophan, tyrosine, valine, phenylacetic acid, α-hydroxyioscaproic acid, 5-amino valeric acid, choline, glycreol-3-phosphate, N-acetylneuraminic acid, uric acid, reduced glutathione, oxidized glutathion, ascorbic acid, and glutamine. In still yet another embodiment, the “reference metabolites” may include one or more of unknown compounds listed in Table 5.

As used herein, the term “sample” or “biological sample” means biological material isolated from a subject. The biological sample may include any biological material suitable for detecting the desired metabolites, and may comprise cellular and/or non-cellular material from the subject. In one embodiment, the sample can be isolated from any suitable gingival crevicular fluid (GCF).

The present invention relates to the differential expression profiles of metabolites in periodontal diseases and methods based upon these differential expression profiles.

I. Differential Expression Profiles

A. Metabolites

The periodontal disease metabolites described herein were discovered using metabolomic profiling techniques. Such metabolomic profiling techniques are described in more detail in the Examples set forth below, as well as in U.S. Pat. No. 7,005,255 and U.S. patent application Ser. Nos. 11/357,732, 10/695,265 (Publication No. 2005/0014132), 11/301,077 (Publication No. 2006/0134676), 11/301,078 (Publication No. 2006/0134677), 11/301,079 (Publication No. 2006/0134678), and 11/405,033, the entire contents of which are hereby incorporated herein by reference.

Tables 1-5 tabulate a series of metabolites which correlate with healthy oral status or periodontal disease.

Although the identities of some of the metabolites and non-metabolite compounds are not known at this time, such identities are not necessary for the identification of the metabolites or non-metabolite compounds in biological samples from subjects, as the “unnamed” compounds have been sufficiently characterized by analytical techniques to allow such identification. The analytical characterization of all such “unnamed” compounds is listed in Table 5. Such “unnamed” metabolites and non-metabolite compounds are designated herein using the nomenclature “Dental” followed by a specific compound number. Table 5 lists a group of unknown metabolites which correlated with healthy oral status or periodontal disease.

B. Expression Profiles

Generally, metabolite profiles were determined for biological samples from human subjects diagnosed with a periodontal disease, as well as from healthy human subjects. The expression profiles for biological samples from periodontal disease subjects were compared to the expression profiles for biological subjects collected front healthy subjects. Those molecules or compounds differentially expressed, including those differentially expressed at a level that is statistically significant, in the expression profiles of periodontal disease samples as compared to non-disease samples were identified.

The experimental design, metabolite expression profiling platforms, statistical methods and analysis, and results are discussed in more detail in the Examples.

II. Methods Based on Metabolite Profiles

In some embodiments, the present invention relates to analytical and diagnostic methods based on the metabolite profiles for periodontal diseases including, but not limited to: methods for the diagnosis of periodontal diseases, methods of monitoring the progression/regression of periodontal diseases, methods of assessing the efficacy of compositions for treating periodontal diseases, methods of treating periodontal diseases, and the like. In one embodiment, the metabolite profiles may be generated from gingival crevicular fluid of a sample.

A. Methods for the Diagnosis of Oral Health or Periodontal Disease

An aspect of the present invention relates to the diagnosis of periodontal disease development. In one embodiment, the diagnosis may be made prior to the appearance of clinical signs of disease development. In one embodiment, the present invention provides for a method for diagnosing oral health in a subject in which a gingival crevicular fluid sample is collected from the subject and a level of one or more metabolites in the gingival crevicular fluid sample is detected. The subject is diagnosed as having periodontal disease or healthy oral status based on the level of the detected metabolite(s). In one such embodiment, the detected metabolite is at least one compound chosen from: a compound generated by amino acid metabolism, a compound generated in urea cycle; a compound generated in glutathion conversion; a compound generated in lipid metabolism; a compound generated in carbohydrate metabolism; a compound generated by nucleic acid metabolism; vitamins; and co-factors. In another embodiment, the detected metabolites may include one or more of compounds listed in Tables 1, 2, 3, 4 and 5. In one embodiment, a diagnosis of periodontal disease corresponds to an up regulation of one or more of the following compounds: inosine, hypoxanthine, xanthine, guanosine, guanine, leucine, isoleucine, lysie, methionine, phenyllalanine, proline, serine, threonine, tryptophan, tyrosine, valine, phenylacetic acid, α-hydroxyioscaproic acid, 5-amino valeric acid, choline, glycreol-3-phosphate, and N-acetylneuraminic acid. In another embodiment, a diagnosis of periodontal disease corresponds to a down regulation of one or more of the following compounds: uric acid, reduced glutathione, oxidized glutathion, ascorbic acid, and glutamine.

In another embodiment, the present invention discloses a method for diagnosing oral health in a subject in which a gingival crevicular fluid sample is collected from the subject and a level of one or more metabolites in the gingival crevicular fluid sample is detected. The levels of detected metabolites in the gingival crevicular fluid sample are compared to a metabolite reference level wherein the metabolite reference level correlates with one or more of the following: periodontal disease or healthy oral status. In one such embodiment, the detected metabolite levels are compared to one of more of the following: periodontal reference levels; and healthy reference levels to aid in diagnosing or to diagnose whether the subject has a periodontal disease or a healthy oral status. In one embodiment, detected levels of the one or more metabolites may be compared using a simple comparison (e.g., a manual comparison). In another embodiment, the detected levels of the one or more metabolites in the biological sample may also be compared using one or more statistical analyses (e.g., t-test, Welch's T-test. Wilcoxon's rank sum test, random forest).

In one such embodiment, the sample can be a crevicular fluid sample obtained from the oral cavity of a subject. In one such embodiment, the detected metabolite may be a compound chosen from: a compound generated by amino acid metabolism, a compound generated in urea cycle; a compound generated in glutathion conversion; a compound generated in lipid metabolism; a compound generated in carbohydrate metabolism; a compound generated by nucleic acid metabolism: vitamins; and co-factors. In another embodiment, the metabolites may include one or more of compounds listed in Tables 1, 2, 3, 4 and 5.

In another embodiment, the comparing step may include comparing the detected metabolite level to periodontal reference levels or healthy reference levels. In one such embodiment, the detected levels of the one or more metabolites in a sample which correspond to the periodontal reference levels maybe one or more of the following: detected levels that are the same as the periodontal reference levels; detected levels that are substantially the same as the periodontal reference levels; detected levels that are above and/or below the minimum and/or maximum of the periodontal reference levels; and/or detected levels that are within the range of the periodontal reference levels. Such detected levels maybe indicative of a diagnosis of periodontal disease in the subject. In another such embodiment, detected levels of the one or more metabolites in a sample which correspond to a healthy reference levels which may be one or more of the following: detected levels that are the same as the healthy reference levels; detected levels that are substantially the same as the healthy reference levels; detected levels that are above and/or below the minimum and/or maximum of the healthy reference levels, and/or detected levels that are within the range of the healthy reference levels. Such levels maybe indicative of a diagnosis of a healthy oral status in the subject.

In one embodiment, detected levels of the one or more metabolites that are differentially expressed (especially at a level that is statistically significant) by the test subject as compared to periodontal reference levels maybe indicative of a diagnosis of periodontal disease in a subject. In another embodiment, detected levels of the one or more metabolites that are differentially expressed (especially at a level that is statistically significant) by the test subject as compared to healthy reference levels maybe indicative of a diagnosis of a healthy oral status in a subject.

In one embodiment, determining levels of combinations of the detected metabolites may: allow greater sensitivity and specificity in diagnosing periodontal disease or healthy oral status; aid in the diagnosis of periodontal disease or healthy oral status; and allow better differentiation between periodontal disease and healthy oral status that may have similar or overlapping metabolites. In one embodiment, ratios of the detected levels of certain metabolites (and non-metabolite compounds) in biological samples may: allow greater sensitivity and specificity in diagnosing a periodontal disease or healthy oral status; aid in the diagnosis of periodontal oral status; and allow better differentiation of healthy oral status, gingivitis oral status or periodontal disease from each other and from other diseases that may have similar or overlapping metabolites.

B. Methods for the Identification of Subjects for Clinical Trials

The invention also pertains to a method for predicting a subject's response, e.g., responder or non-responder, to using a therapeutic agent for periodontal disease or a lack of using a therapeutic agent for periodontal disease.

As used herein, a “responder” means a subject which shows: a decrease in metabolite levels that correlate with periodontal disease; an increase in metabolite levels that correlate with periodontal disease; a decrease in metabolite levels that correlate with healthy oral status; and an increase in metabolite levels that correlate with healthy oral status.

As used herein, a “non-responder” means a subject which shows no change in metabolite levels that correlate with periodontal disease or healthy oral status.

1. Method for Identifying a Responder or Non-Responder to a Therapeutic Agent

One aspect of the present invention relates to a method that may be used to determine whether a subject is suitable to participate in a clinical trial of test therapeutic agent(s) for treatment of periodontal disease. During the course of clinical trials for test therapeutic agents, some subjects may not show evidence of responding to the test therapeutic agent while following a test protocol during the time period of the trial, i.e., non-responder. For example, an oral examination of the subject may show no changes in symptoms such as: swollen, red or bleeding gums; receding gum line; loose or separated teeth, bad breath, etc. A non-responder subject is not a desirable participate in a clinical trial because limited if any information may be obtained from the non-responder subject's participation in the trial. It would be advantageous to identify a non-responder at the start of the clinical trial or during early stages of the clinical trial so to eliminate the non-responder subject from the group of test subjects.

The method of the invention, described below, provides for identification of responder subjects and non-responder subjects to a therapeutic agent. In one embodiment, the method includes generating a metabolite profile of a gingival crevicular fluid sample collected from a test subject while following a test protocol, wherein the metabolite profile includes the metabolite identity and metabolite level and comparing the metabolite profile of the test subject to a reference metabolite profile. The reference metabolite profile may include one or more of: a reference responder metabolite profile and a reference non-responder metabolite profile. The results of the comparison can be used to identify the test subject as responder or non-responder to therapeutic agent. In one embodiment, the comparison may be made using a simple comparison (e.g., a manual comparison), in another embodiment, the comparison may be made using one or more statistical analyses (e.g., t-test, Welch's T-test, Wilcoxon's rank sum test, random forest).

In one such embodiment, the reference responder metabolite profile may be generated from the gingival crevicular fluid sample of one or more reference responder subjects who showed regression of periodontal disease, or prevention of periodontal disease when using a dentifrice containing a standard therapeutic agent according to a standard care protocol during the number of days of the standard care protocol. In another embodiment, the reference non-responder metabolite profile may be generated from the gingival crevicular fluid sample of one or more reference non-responder subjects who showed no change in periodontal disease when using a dentifrice containing a standard therapeutic agent according to a standard care protocol during the number of days of the standard care protocol. The standard care protocol may include instructions such as brushing duration, number of times per day, number of days, use of other oral care products, etc.

The reference responder subject and/or reference non-responder subject may be one or more of the following: a reference responder subject and/or reference non-responder subject identified as having a healthy oral status based on clinical evaluation by a dental professional; a reference responder subject and/or reference non-responder subject identified as having gingivitis based on clinical evaluation by dental professional; and a reference responder subject and/or reference non-responder subject identified as having periodontal disease based on clinical evaluation by a dental professional.

In one embodiment, the standard therapeutic agent down-regulates at least one member chose from: inosine, hypoxanthine, xanthine, guanosine, guanine, leucine, isoleucine, lysie, methionine, phenyllalanine, proline, serine, threonine, tryptophan, tyrosine, valine, phenylacetic acid, α-hydroxyioscaproic acid, 5-amino valeric acid, choline, glycreol-3-phosphate, and N-acetylneuraminic acid for the reference subject. In another embodiment, the standard therapeutic agent up-regulates at least one member chose from: uric acid, reduced glutathione, oxidized glutathion, ascorbic acid, and glutamine. In yet another embodiment, the standard therapeutic agent up-regulates or down-regulates at least one member of unknowns listed in Table 5.

In one embodiment, the test subject's metabolite profile may be generated from one or more gingival crevicular fluid samples which may be collected from the test subject in a single collecting step prior to initiating a standard care protocol. In another embodiment, the test subject's metabolite profile may be collected from one or more gingival crevicular fluid samples which may be obtained from the test subject in a single collecting step after the test subject has followed a standard care protocol for a prescribed number of days. In one embodiment, the test subject's metabolite profile may be determined from one or more gingival crevicular fluid samples which may be collected from the test subject in multiple collecting steps, each collecting step occurring on a different day after the test subject has followed a standard care protocol for a prescribed number of days.

In one such embodiment, the metabolite identity of the test subject may correspond to a compound chosen from: a compound generated by amino acid metabolism, a compound generated in urea cycle; a compound generated in glutathion conversion; a compound generated in lipid metabolism; a compound generated in carbohydrate metabolism; a compound generated by nucleic acid metabolism: vitamins; and co-factors. In another embodiment, the metabolite identity of the test subject may include one or more of compounds listed in Tables 1, 2, 3, 4 and 5.

In another embodiment, the comparing step may include comparing the metabolite level of the test subject to reference responder levels and reference non-responder levels. In one such embodiment, the metabolite levels of the test subject which may correspond to reference responder metabolite levels maybe one or more of the following: metabolite levels that are the same as the reference responder metabolite levels; metabolite levels that are substantially the same as the reference responder metabolite levels; metabolite levels that are above and/or below the minimum and/or maximum of the reference responder metabolite levels; and/or metabolite levels that are within the range of the reference responder metabolite levels. Such metabolite levels maybe indicative of an identification of the test subject as a responder to therapeutic agents. In another such embodiment, metabolite levels of the test subject which may correspond to reference non-responder metabolite levels may be one or more of the following: metabolite levels that are the same as the reference non-responder metabolite levels; metabolite levels that are substantially the same as the reference non-responder metabolite levels; metabolite levels that are above and/or below the minimum and/or maximum of the reference non-responder metabolite levels, and/or metabolite levels that are within the range of the reference non-responder metabolite levels. Such levels maybe indicative of a diagnosis of an identification of the test subject as a non-responder to therapeutic agents.

In one embodiment, metabolite levels of the one or more metabolites that are differentially expressed (especially at a level that is statistically significant) of the test subject as compared to responder reference levels maybe indicative of an identification of a subject as a responder to therapeutic agents. In another embodiment, metabolite levels of the one or more metabolites that are differentially expressed (especially at a level that is statistically significant) of the test subject as compared to non-responder reference levels maybe indicative of an identification of a subject as a non-responder to therapeutic agents.

2. Method for Identifying a Responder or Non-Responder to Periodontal Disease Development

According to one aspect, the present invention provides for a method which may be used to determine whether a subject is suitable to participate in a clinical trial of test therapeutic agent(s) for treatment of periodontal disease, wherein the subject is susceptible to periodontal disease development. In some instances, a clinical trial of a test therapeutic agent may be conducted by first generating gingivitis in one or more sections of teeth in a subject's oral cavity. This may be accomplished by using a non-care protocol where a shield is placed over the one or more sections of teeth so that the shielded section(s) of teeth do not receive any form of oral hygiene. Subsequent to gingivitis development, the subject may use a test therapeutic agent according to a care protocol to treat the gingivitis. In some instances, a subject may not show evidence of gingivitis during the time period of the non-care protocol.

In one embodiment of the present invention, a method includes generating a metabolite profile of the gingival crevicular fluid sample collected from a test subject while following a standard non-care protocol, wherein the metabolite profile includes the metabolite identity and metabolite level and comparing the metabolite profile of the test subject to a reference metabolite profile. The reference metabolite profile may include one or more of: a reference responder metabolite profile and a reference non-responder metabolite profile. The results of the comparison can be used to identify the test subject as responder or non-responder to periodontal disease development. In one embodiment, the comparison may be made using a simple comparison a manual comparison). In another embodiment, the comparison may be made using one or more statistical analyses (e.g., t-test, Welch's T-test, Wilcoxon's rank sum test, random forest).

In one such embodiment, the reference responder metabolite profile can be obtained from the crevicular fluid sample of one or more reference responder subjects who followed a non-care standard protocol and developed periodontal disease during the length of time of the protocol. In another such embodiment, the reference non-responder metabolite profile may be obtained from the crevicular fluid sample of one or more reference non-responder subjects who failed to develop periodontal disease during the length of time of a standard non-care protocol. The standard no-oral care protocol may include one or more of the following: absence of brushing for a prescribe number of days, wearing a shield over one or more sections of teeth while caring for the other sections of teeth according to a protocol which describes brushing duration, number of brushing times per day, number of days, and the use of mechanical oral hygiene devices.

In one embodiment, the standard non-care protocol up regulates at least one member chose from: inosine, hypoxanthine, xanthine, guanosine, guanine, leucine, isoleucine, lysie, methionine, phenyllalanine, proline, serine, threonine, tryptophan, tyrosine, valine, phenylacetic acid, α-hydroxyioscaproic acid, 5-amino valeric acid, choline, glycreol-3-phosphate, and N-acetylneuraminic acid for the reference subject. In another embodiment, the standard non-care protocol down-regulates at least one member chose from: uric acid, reduced glutathione, oxidized glutathion, ascorbic acid, and glutamine for the reference subject. In yet another embodiment, the standard therapeutic agent up-regulates or down-regulates at least one member of unknowns listed in Table 5.

In one embodiment, the test subject's metabolite profile may be generated from one or more gingival crevicular fluid samples which may be obtained from the test subject in a single collecting step prior to initiating a standard non-care protocol. In another embodiment, the test subject's metabolite profile may be generated from one or more gingival crevicular fluid samples which may be obtained from the test subject in a single collecting step after the test subject has followed a standard non-care protocol for a prescribed number of days. In one embodiment, the test subject's metabolite profile may be determined from one or more gingival crevicular fluid samples which may be obtained from the test subject in multiple collecting steps, each collecting step occurring on a different day after the test subject has followed a standard non-care protocol for a prescribed number of days.

In one such embodiment, the metabolite identity of the test subject may correspond to a compound chosen from: a compound generated by amino acid metabolism, a compound generated in urea cycle; a compound generated in glutathion conversion; a compound generated in lipid metabolism; a compound generated in carbohydrate metabolism; a compound generated by nucleic acid metabolism; vitamins; and co-factors. In another embodiment, the metabolite identity of the test subject may include one or more of compounds listed in Tables 1, 2, 3, 4 and 5.

In another embodiment, the comparing step may include comparing the metabolite level to reference responder levels and reference non-responder levels. In one such embodiment, the metabolite levels of the test subject which may correspond to reference responder metabolite levels maybe one or more of the following: metabolite levels that are the same as the reference responder metabolite levels; metabolite levels that are substantially the same as the reference responder metabolite levels; metabolite levels that are above and/or below the minimum and/or maximum of the reference responder metabolite levels; and/or metabolite levels that are within the range of the reference responder metabolite levels. Such metabolite levels maybe indicative of an identification of the test subject as a responder to a non-care protocol, e.g., development of gingivitis and/or periodontal disease. In another such embodiment, metabolite levels of the test subject which may correspond to reference non-responder metabolite levels may be one or more of the following: metabolite levels that are the same as the reference non-responder metabolite levels; metabolite levels that are substantially the same as the reference non-responder metabolite levels; metabolite levels that are above and/or below the minimum and/or maximum of the reference non-responder metabolite levels, and/or metabolite levels that are within the range of the reference non-responder metabolite levels. Such levels maybe indicative of a diagnosis of an identification of the test subject as a non-responder responder to non-care protocol, e.g. fails to develop gingivitis and/or periodontal disease.

In one embodiment, metabolite levels of the one or more metabolites that are differentially expressed (especially at a level that is statistically significant) by the test subject as compared to responder reference levels maybe indicative of an identification of a subject as a responder to a non-care protocol. In another embodiment, metabolite levels of the one or more metabolites that are differentially expressed (especially at a level that is statistically significant) by the test subject as compared to non-responder reference levels maybe indicative of an identification of a subject as a non-responder to a non-care protocol.

C. Methods for Evaluating the Efficiency of a Test Compound in a Dentifrice

The present invention also provides for a method of determining an efficiency of a test compound useful in treating periodontal disease development in a mammal. In one embodiment, the method includes detecting a post-treatment metabolite level from a gingival crevicular fluid sample collected from a subject after treatment with a test compound. The post-treatment metabolite level may be compared to one or more of the following: pre-treatment metabolite levels of the subject: periodontal reference levels and healthy reference levels. In one such embodiment, the method may include the step of determining whether the test compound down-regulates at least one member chose from: inosine, hypoxanthine, xanthine, guanosine, guanine, leucine, isoleucine, lysie, methionine, phenyllalanine, proline, serine, threonine, tryptophan, tyrosine, valine, phenylacetic acid, α-hydroxyioscaproic acid, 5-amino valeric acid, choline, glycreol-3-phosphate, and N-acetylneuraminic acid. In another such embodiment, the method may include the step of determining whether the test compound up-regulates at least one member chose from: uric acid, reduced glutathione, oxidized glutathion, ascorbic acid, and glutamine. In another embodiment, the method may include the step of determining whether the test compound up-regulates or down-regulates at least one member of unknowns listed in Table 5.

In another embodiment, the pre-treatment metabolite level may be obtained by detecting a pretreatment metabolite level of a first gingival crevicular fluid sample collected from the subject at a first point in time. A dentifrice containing the test compound may be applied to an oral cavity of the subject according to a prescribed protocol. At a second point in time after applying the dentriface, the post-treatment metabolite level of a second gingival crevicular fluid sample is detected. The pretreatment metabolite level may be compared to the post-treatment metabolite level. Based on the comparison, the efficiency of the test compound may be determined. In one such embodiment, a decrease in post-treatment metabolite levels compared to pre-treatment metabolite levels may be indicative of the test compound having efficacy to treat periodontal disease. In another such embodiment, an increase in post-treatment metabolite levels compared to pre-treatment metabolite levels may be indicative of the test compound having efficacy to treat periodontal disease. In still another such embodiment, the absence of a decrease or increase in post-treatment metabolite levels may be indicative that the test compound lacks efficacy to treat periodontal disease.

In another embodiment, the post-treatment metabolite levels may be compared to one or more of: periodontal disease reference levels and healthy oral status reference levels, one embodiment, the comparison may be made using a simple comparison (e.g., a manual comparison). In another embodiment, the comparison may be made using one or more statistical analyses (e.g., t-test, Welch's T-test, Wilcoxon's rank sum test, random forest). In one such embodiment, the results of the comparison may be indicative of the efficacy of the test compound when the post-treatment metabolite levels are one or more of the following: post-treatment metabolite levels that are the same as the periodontal reference levels; post-treatment metabolite levels are substantially the same as the periodontal reference levels; post-treatment metabolite levels are above and/or below the minimum and/or maximum of the periodontal reference levels; and/or post-treatment metabolite levels are within the range of the periodontal reference levels. In another such embodiment, the results of the comparison may be indicative of the efficacy of the test compound when the post-treatment metabolite levels are one or more of the following: post-treatment metabolite levels that are the same as the healthy reference levels; post-treatment metabolite levels are substantially the same as the healthy reference levels; post-treatment metabolite levels are above and/or below the minimum and/or maximum of the healthy reference levels; and/or post-treatment metabolite levels are within the range of the healthy reference levels.

The invention further provides for a method of identifying a test compound useful in treating periodontal disease in a mammal by contacting a cell with the test compound and determining whether the test compound down-regulates at least one member chose from: inosine, hypoxanthine, xanthine, guanosine, guanine, leucine, isoleucine, lysie, methionine, phenyllalanine, proline, serine, threonine, tryptophan, tyrosine, valine, phenylacetic acid, α-hydroxyioscaproic acid, 5-amino valeric acid, choline, glycreol-3-phosphate, and N-acetylneuraminic acid.

The invention further yet provides for a method of identifying a test compound useful in treating periodontal disease in a mammal by contacting a cell with the test compound and determining whether the test compound up-regulates at least one member chose from: uric acid, reduced glutathione, oxidized glutathion, ascorbic acid, and glutamine.

The invention further yet provides for a method of identifying a test compound useful in treating periodontal disease in a mammal by contacting a cell with the test compound and determining whether the test compound up-regulates or down-regulates at least one member of unknowns listed in Table 5.

D. Oral Care Test Kit

The present invention further provides for an oral care test kit which may provide the user an indication of the user's oral health status. The kit may include one or more gingivitis crevicular fluid collection strips and a diagnosis of the subject's oral health status. The gingivitis crevicular fluid collection strips may be used for collecting a gingival crevicular fluid sample and for recovery of metabolites contained in the gingival crevicular fluid sample. The diagnosis of a subject's oral health may be based on the methods of this invention. In one such embodiment, the kit may include instructions for using the gingivitis crevicular fluid collection strips to collect a sample. In another such embodiment, the kit may include directions for sending the gingivitis crevicular fluid collection strips with collected fluid to a test site.

E. Dentifrice Composition

The present invention further provides for a dentifrice composition. The composition may include an effective amount of an oral health metabolite therapeutic agent. The therapeutic agent effects a change in metabolite levels over a time period of at least one month wherein the change metabolite level is greater than a corresponding change in metabolite reference levels affected by a control dentifrice composition. In one embodiment, the change in metabolite level is greater than 1% than corresponding change in metabolite reference levels affected by a control dentifrice composition. In another embodiment, the change in metabolite level is greater than 5% than corresponding change in metabolite reference levels affected by a control dentifrice composition. In yet another embodiment, the change in metabolite level is greater than 20% than corresponding change in metabolite reference levels affected by a control dentifrice composition. In one embodiment, the control dentifrice is substantially tree of a standard therapeutic agent. In another embodiment, the control dentifrice contains a standard therapeutic agent. In another embodiment, the control dentifrice contains Triclosan. The control dentifrice also may include ingredients typically found in dentifrice compositions as described in U.S. Pat. No. 7,402,416 which is incorporated herein reference in its entirety.

F. Metabolite Indicating Compositions

In another aspect, the present invention provides for an oral composition for indicating the presence of metabolites indicative of periodontal disease. The oral composition may be included within a dentifrice such as tooth paste, gel, mouth wash, dental floss, powder, gum adhering strip, tooth brush, etc. The oral composition may include metabolite indicating composition. In one embodiment, the metabolite indicating composition may indicate the presence of one or more compound listed in Tables 1, 2, 3, 4 and 5. In another embodiment, the metabolite indicating composition may be present a user discernable indicator upon up regulation of one or more of the following: inosine, hypoxanthine, xanthine, guanosine, guanine, leucine, isoleucine, lysie, methionine, phenyllalanine, proline, serine, threonine, tryptophan, tyrosine, valine, phenylacetic acid, α-hydroxyioscaproic acid, 5-amino valeric acid, choline, glycreol-3-phosphate, and N-acetylneuraminic acid. In another embodiment, the metabolite indicating composition may be down regulation of one or more of the following: uric acid, reduced glutathione, oxidized glutathion, ascorbic acid, and glutamine. In one embodiment, the metabolite indicating composition may result in a user discernable indicator when exposed to one or more of the compounds listed in Tables 1, 2, 3, 4 and 5. In one embodiment, the metabolite indicating composition may result in a color change of the dentifrice when exposed to one or more of the compounds listed in Tables 1, 2, 3, 4 and 5.

In one embodiment, the dentifrice may include a gel which adheres to the gingival margin and contains a metabolite indicating composition. In one such embodiment, the gel may be applied to one or more teeth quadrants of a subject's oral cavity wherein the metabolite indicating composition may result in appearance of a user discernable indicator when exposed to one or more of the compounds listed in Tables 1, 2, 3, 4 and 5. In one embodiment, the user discernable indicator may correspond to a change in color.

In another embodiment, the dentifrice may include dental floss coated with a metabolite indicating composition. In one such embodiment, the metabolite coated dental floss may be passed between adjacent teeth of a user, wherein the metabolite indicating composition may result in the floss showing a user discernable indicator when exposed to one or more of the compounds listed in Tables 1, 2, 3, 4 and 5. In one embodiment, the user discernable indicator may correspond to the floss showing a change in color.

In yet another embodiment, the dentifrice may include mouth wash containing a metabolite indicating composition. In one such embodiment, a user may contact its teeth with the mouth wash, wherein the metabolite indicating composition may result in an appearance of a user discernable indicator of the mouthwash when exposed to one or more of the compounds listed in Tables 1, 2, 3, 4 and 5. In one embodiment, the user discernable indicator may correspond to a change in color of the mouthwash.

### EXAMPLES

The following examples further describe and demonstrate some embodiments within the scope of the present invention. These examples are given solely for the purpose of illustration and are not to be construed as limitations of the present invention as many variations thereof are possible without departing from the scope and spirit of the present invention.

I. Experimental Procedures

A. Experimental Design and Patients

Twenty-two (22) chronic periodontitis subjects (41% males) of 33 to 67 years of age (53±11) were selected from research volunteers at the Forsyth Institute Dental Clinic. Subjects had at least 20 natural uncrowned teeth, and ≧8 sites with pocket depth ≧5 mm and clinical attachment level (CAL)≧3 mm. Subjects had no known history of allergy to the dentifrice for the washout period (see below). Exclusion criteria included: presence of orthodontic appliances; abnormal salivary function; use of prescription drugs; use of antibiotics 1 month prior to or during the study; use of any over the counter medications other than analgesics; daily use of vitamin supplements; 5 or more decayed dental sites; diseases of the soft or hard oral tissues and systemic conditions. The study protocol was approved by The Forsyth Institute's Institutional Review Board and all study subjects signed an informed consent form prior to enrollment. At first visit, subjects received a tube of Colgate Regular dentifrice and a toothbrush and were instructed to use the product for a minimum of 1 week (washout period) prior to their sampling visit. Other mechanical oral hygiene devices were allowed during this washout period, but no other oral care products.

B. Crevicular Fluid (GCF) Sample Collection

Following isolation of sites with cotton rods to prevent contamination with saliva, supragingival plaque was removed with a curette, sites gently air dried and GCF samples obtained from 3 different site categories. For each subject, 6 healthy (H: PD≦3 mm and no bleeding on probing [BOP]); 6 gingivitis (C; PD≦3 mm and BOP): and 3 periodontitis (P: PD≧5 mm and BOP) sites were sampled. GCF samples were collected from each site using filter strips (Periopaper® Interstate Drug Exchange, Amityville, N.Y.) gently inserted into the orifice of the periodontal pocket. Periopaper strips were kept in place for 30 seconds and the GCF volume collected was determined using a pre-calibrated. Periotron 8000® (Oratlow Inc., Plainview, N.Y.). Samples from each subject were pooled into different site categories, placed in separate Eppendorf tubes and stored at −80° C. until assay.

C. Metabolite Expression Profiling Technology

Metabolite expression profiling technology was performed as described previously (Lawton et al., 2008). In summary, a four-step sequential extraction procedure was employed to recover metabolites from the GCF collection strips. The extracts were analyzed by GC/MS and LC/MS. Chromatographic separation followed by full scan mass spectra was carried out to record and quantify all detectable ions presented in the samples. Metabolites with known chemical structure were identified by matching the ions' chromatographic retention index and mass spectra fragmentation signatures with reference library entries created from authentic standard metabolites under the identical analytical procedure as the experimental samples. For ions that were not covered by the standards, additional library entries were added based on their unique ion signatures (chromatographic and mass spectral). After this, these ions can be routinely detected and quantified.

D. Statistical Analysis

Data were normalized using the GSF volume recorded by Periotron®. ANOVA and T-tests were performed to compare data obtained from the healthy, gingivitis and periodontitis sites. Log transformation was applied to the observed relative concentrations for each biochemical.

E. Results

A total of 330 individual samples were collected, resulting in 66 pooled samples. The GCF samples ranged from 0.01 μl to 1.15 μl. The mean volumes in μl (±SD) for the healthy, gin gingivitis and periodontitis site, categories 0.18±0.10, 0.25±0.13 and 0.42±0.19, respectively.

The samples were analyzed in metabolite profiling platforms by Metabolon, Inc. The relative quantitated values for the compounds were then adjusted according to sample volume. Two hundred twenty eight (228) metabolites were detected, of which one hundred three (103) matched known chemical structures in the Metabolon chemical reference library. Matched pair T-test was used to analyze the differences among the healthy, gingivitis and periodontitis sites. Approximately 50% of the detected metabolites showed altered levels among the three sites (p<0.05). The metabolites matching known chemical structures were mapped into their respective general biochemical pathways. ANOVA analysis did not produce a list of metabolites different from the t-tests (data not shown). For the majority of metabolites with altered concentrations, the levels at gingivitis sites resided between the levels at healthy and periodontitis sites, suggesting that the metabolic changes induced by gingivitis are continuum to those of periodontitis.

1. Nucleic Acids

Bacteria degrade and metabolize host nucleic acids. Elevated levels of nucleic acids degradation end products and intermediates suggest acceleration of the nucleic acid degradation pathways by bacterial infection. The end product of the degradation of the purine nucleotides adenosine monophosphate (AMP) and guanosine monophosphate (GMP) is uric acid. The intermediates in the pathway include inosine, hypoxanthine, xanthine, guanosine and guanine. Referring to Table 1 below, the differential expression profiles for the purine degradation pathway intermediates are tabulated.

Regarding inosine, in the present study, there was a 1.22 fold increase in inosine levels between gingivitis and healthy subjects, and a 1.63 fold increase between periodontitis and healthy subjects. Regarding hypoxanthine, there was a 1.27 fold increase in hypoxanthine levels between gingivitis and healthy subjects, and a 2.65 fold increase between periodontitis and healthy subjects. Regarding xanthine, there was a 1.15 fold increase in xanthine levels between gingivitis and healthy subjects, and a 2.15 fold increase between periodontitis and healthy subjects. Regarding guanosine, there was a 1.02 fold increase in guanosine levels between gingivitis and healthy subjects, and a 1.35 fold increase between periodontitis and healthy subjects. Regarding guanine, there was a 1.22 fold increase in guanine levels between gingivitis and healthy subjects, and a 1.66 fold increase between periodontitis and healthy subjects. The increased expression—“up-regulation”—of these intermediates at the disease sites in the present study indicates accelerated metabolic flux of the purine degradation pathway due to bacterial infection.

Contrary to the increases of the intermediates, the levels of the end product uric acid decreased at the disease sites. There was a 0.92 fold decrease in uric acid levels between gingivitis and healthy subjects, and a 0.70 fold decrease between periodontitis and healthy subjects. However, uric acid is a known cellular antioxidant, and as described in the next section, there is clear evidence that oxidative stress was also intensified at the disease sites. The decrease of uric acid could be the result of its depletion upon scavenging free radicals. Further, the consecutive steps of conversion of hypoxanthine to xanthine and then to uric acid are both catalyzed by xanthine oxidase. The reactions are coupled with a reduction of oxygen to generate superoxides in the form of O₂⁻ and H₂O₂. The changes with the purine degradation pathway observed here indicate that increased reactive oxygen species (ROS) production is a significant consequence derived from up-regulation of this pathway by the periodontal diseases.

TABLE 1
Nucleic Acids Degradation Pathway Compounds

| | | | | H vs. G | | H vs. P | | G vs. P | |
|---|---|---|---|---|---|---|---|---|---|
| | MEAN | | | p | q | p | q | p | q |
| COMPOUND | H | G | P | value | value | value | value | value | value |
| Inosine | 1 | 1.22 | 1.63 | 0.2621 | 0.2466 | 0 | 0.0002 | 0.0869 | 0.0715 |
| Hypoxanthine | 0.96 | 1.22 | 2.54 | 0.0179 | 0.0745 | 0 | 0 | 0 | 0.0017 |
| Xanthine | 1.09 | 1.25 | 2.34 | 0.3144 | 0.275 | 0 | 0 | 0.0001 | 0.0017 |
| Guanosine | 0.84 | 0.86 | 1.13 | 0.0432 | 0.1258 | 0.0123 | 0.0126 | 0.3739 | 0.1947 |
| Guanine | 0.77 | 0.94 | 1.28 | 0.0692 | 0.1447 | 0.0121 | 0.0126 | 0.1255 | 0.0891 |
| Uric acid | 1.21 | 1.11 | 0.85 | 0.0536 | 0.138 | 0.0005 | 0.0011 | 0.0398 | 0.046 |
| Uridine | 0.83 | 1 | 1.51 | 0.1479 | 0.1875 | 0.032 | 0.0249 | 0.1122 | 0.0816 |

The end product of the degradation of the pyrimidine nucleotides cytidine monophosphate (CMP) and uridine monophosphate (UMP) is uracil. An intermediate in the pathway is uridine, and its up-regulation at the disease sites indicates accelerated metabolic flux of the pyrimidine degradation pathway due to bacterial infection. There was a 1.20 fold increase in uridine levels between gingivitis and healthy subjects, and a 1.82 fold increase between periodontitis and healthy subjects. The differential expression profile for uridine is also summarized above in Table 1.

2. Anti-Oxidants

Bacterial infections induce oxidative stress and decreased levels of anti-oxidants. Glutathione plays a central role in cellular defense against ROS (including oxygen ions, free radicals and peroxides) and xenobiotics. In the present study, the levels of both reduced and oxidized glutathione were decreased at the gingivitis and periodontitis sites. The decreased level of glutathione and related metabolites in the glutathione biosynthesis pathway indicates an increased oxidative stress environment and a decreased ability for glutathione production resulting from bacterial infection. There was a 0.65 fold decrease in reduced glutathione levels between gingivitis and healthy subjects, and a 0.35 fold decrease between periodontitis and healthy subjects. There was a 0.75 fold decrease in oxidized glutathione levels between gingivitis and healthy subjects, and a 0.47 fold decrease between periodontitis and healthy subjects.

In addition, two other major cellular anti-oxidants, ascorbic acid and uric acid, were also decreased at the disease sites. There was a 0.87 fold decrease in ascorbic acid levels between gingivitis and healthy subjects, and a 0.39 fold decrease between periodontitis and healthy subjects. As discussed above, there was a 0.92 fold decrease in uric acid levels between gingivitis and healthy subjects, and a 0.70 fold decrease between periodontitis and healthy subjects. The changed expression profiles of these metabolites clearly demonstrate an oxidative stress environment in disease conditions, and are summarized below in Table 2.

TABLE 2
Anti-Oxidant Compounds

| | | | | H vs. G | | H vs. P | | G vs. P | |
|---|---|---|---|---|---|---|---|---|---|
| | MEAN | | | p | q | p | q | p | q |
| COMPOUND | H | G | P | value | value | value | value | value | value |
| Glutathione-red. | 2.34 | 1.51 | 0.81 | 0.3303 | 0.2821 | 0.0319 | 0.0249 | 0.0366 | 0.0456 |
| Glutathione-ox. | 1.39 | 1.04 | 0.66 | 0.6432 | 0.3963 | 0.0205 | 0.0183 | 0.0023 | 0.0070 |
| Ascorbic acid | 1.32 | 1.15 | 0.51 | 0.0979 | 0.1516 | 0.0035 | 0.0043 | 0.051 | 0.0523 |
| Uric acid | 1.21 | 1.11 | 0.85 | 0.0536 | 0.138 | 0.0005 | 0.0011 | 0.0398 | 0.046 |

3. Amino Acids

Consistent with host protein degradation due to tissue damage, the expression levels of a variety of free amino acids and amino acid metabolites were increased by the periodontal diseases. Referring to Table 3 below, the differential expression profiles for the purine degradation pathway intermediates are tabulated.

Regarding isoleucine, in the present study, there was a 1.21 fold increase in isoleucine levels between gingivitis and healthy subjects, and a 1.92 fold increase between periodontitis and healthy subjects. Regarding leucine, there was a 1.12 fold increase in leucine levels between gingivitis and healthy subjects, and a 2.02 fold increase between periodontitis and healthy subjects. Regarding lysine, there was a 1.2 fold increase in lysine levels between gingivitis and healthy subjects, and a 2.79 fold increase between periodontitis and healthy subjects. Regarding phenylalanine, there was a 1.09 fold increase in phenylalanine levels between gingivitis and healthy subjects, and a 1.61 fold increase between periodontitis and healthy subjects. Regarding tyrosine, there was a 1.04 fold increase in tyrosine levels between gingivitis and healthy subjects, and a 1.41 fold increase between periodontitis and healthy subjects. The up-regulation of these amino acids at the disease sites in the present study indicates degradation of host proteins by bacteria.

In addition, putrescine and cadaverine (1,5-diaminopentane), two polyamines and the end products of amino acid degradation, were found to be up-regulated by the periodontal diseases. Regarding putrescine, there was a 1.42 fold increase in putrescine levels between gingivitis and healthy subjects, and a 2.75 fold increase between periodontitis and healthy subjects. Regarding cadaverine, there was a 1.43 fold increase in cadaverine levels between gingivitis and healthy subjects, and a 2.88 fold increase between periodontitis and healthy subjects. While putrescine can be produced by both the mammalian and bacterial pathways, cadaverine is almost exclusively of bacterial origin (Fothergill and Guest, 1977). Cadaverine is synthesized from lysine by bacterial lysine decarboxylase, and elevated expression levels may indicate degrees of bacterial infection.

The only exception to the increased expression of amino acids in the present study was (glutamine, which exhibited a clear decrease with disease progression. There was a 0.84 fold decrease in glutamine levels between gingivitis and healthy subjects, and a 0.60 fold decrease between periodontitis and healthy subjects. One possible explanation is that glutamine was rapidly consumed by the bacteria as the main nitrogen source. Another nitrogen currying amino acid, glutamate, also showed decreases at the disease, although its changes were above the statistical cutoff (data not shown). Another explanation is that in addition to its presentation in proteins, glutamine presents in significant amounts as a free glutamine di-peptide. The bacteria P. ginivalis has been reported to metabolize peptides—rather than single amino acids—and this decrease of free glutamine is consistent with significant utilization of di-peptides by bacteria prior to their degradation to single amino acids.

TABLE 3
Ammo Acids

| | | | | H vs. G | | H vs. P | | G vs. P | |
|---|---|---|---|---|---|---|---|---|---|
| | MEAN | | | p | q | p | q | p | q |
| COMPOUND | H | G | P | value | value | value | value | value | value |
| Isoleucine | 0.91 | 1.1 | 1.75 | 0.0688 | 0.1447 | 0 | 0.0002 | 0.0195 | 0.0283 |
| Leucine | 1.01 | 1.13 | 2.04 | 0.0802 | 0.1447 | 0 | 0.0002 | 0.0054 | 0.0121 |
| Lysine | 1.00 | 1.2 | 2.79 | 0.1628 | 0.197 | 0 | 0 | 0.0002 | 0.0021 |
| Phenylalanine | 0.98 | 1.07 | 1.58 | 0.1005 | 0.1516 | 0 | 0.0002 | 0.0131 | 0.0215 |
| Tyrosine | 1.01 | 1.05 | 1.42 | 0.375 | 0.2853 | 0.0013 | 0.0021 | 0.0166 | 0.0253 |
| Putrescrine | 0.81 | 1.15 | 2.23 | 0.0126 | 0.0685 | 0 | 0.0001 | 0.0009 | 0.0042 |
| Cadaverine | 0.75 | 1.07 | 2.16 | 0.0051 | 0.0549 | 0.0003 | 0.001 | 0.0616 | 0.0597 |
| Glutamine | 1.32 | 1.11 | 0.79 | 0.2167 | 0.2257 | 0.0033 | 0.0043 | 0.0027 | 0.0075 |
| 4-guanidino- | 0.70 | 1.28 | 1.68 | 0.0057 | 0.0549 | 0.0019 | 0.0028 | 0.6566 | 0.2792 |
| butanoic acid | | | | | | | | | |

4. Urea Cycle

The degradation of proteins into amino acids releases ammonia that must then be converted by organisms into less toxic nitrogen forms. In humans, the urea cycle functions to convert ammonia into urea and other end products. Urea cycle intermediates and end products, including putrescine and 4-guanidinobutanoic acid, were significantly up-regulated. Referring to Table 3 above, the differential expression profiles for the urea pathway intermediates are tabulated. Regarding putrescine, as discussed above, there was a 1.42 fold increase in putrescine levels between gingivitis and healthy subjects, and a 2.75 fold increase between periodontitis and healthy subjects. Regarding 4-guanidinobutanoic acid, in the present study, there was a 1.83 fold increase in 4-guanidinobutanoic acid levels between gingivitis and healthy subjects, and a 2.33 fold increase between periodontitis and healthy subjects.

5. Carbohydrates

The expression changes of a variety of sugars and carbohydrate metabolites further illustrate the interactions between the host tissue and bacteria. Referring Table 4 below, the decreased levels of tri- and di-saccharides including maltotriose, maltose and maltotriitol, in the disease sites could be the result of bacterial consumption of these dietary nutrients in circulation. There was a 0.90 fold decrease in maltotriose levels between gingivitis and healthy subjects, and a 0.59 told decrease between periodontitis and healthy subjects. There was a 0.93 fold decrease in maltose levels between gingivitis and healthy subjects, and a 0.72 fold decrease between periodontitis and healthy subjects. There was a 0.86 fold decrease in maltotriiol levels between gingivitis and healthy subjects, and a 0.43 fold decrease between periodontitis and healthy subjects.

The degradation of these tri- and di-saccharides results in increased levels of the end-product, glucose. Referring to glucose, there was a 1.35 fold increase in glucose levels between gingivitis and healthy subjects, and a 1.96 fold increase between periodontitis and healthy subjects. Increased glucose, which is highly regulated by biochemical pathways, thereby results in up-regulation of the Kreb's cycle and the increased expression of intermediates including α-ketoglutarate. There was a 1.65 fold increase in α-ketoglutarate levels between gingivitis and healthy subjects, and a 3.15 fold increase between periodontitis and healthy subjects.

TABLE 4
Carbohydrates

| | | | | H vs. G | | H vs. P | | G vs. P | |
|---|---|---|---|---|---|---|---|---|---|
| | MEAN | | | p | q | p | q | p | q |
| COMPOUND | H | G | P | value | value | value | value | value | value |
| Maltotriose | 1.35 | 1.22 | 0.74 | 0.5129 | 0.3566 | 0.008 | 0.0015 | 0.0007 | 0.0038 |
| Maltose | 1.2 | 1.11 | 0.86 | 0.7274 | 0.4214 | 0.0203 | 0.0183 | 0.0103 | 0.0178 |
| Maltotriiol | 1.39 | 1.2 | 0.63 | 0.5355 | 0.3621 | 0.0002 | 0.0006 | 0.0001 | 0.0017 |
| Glucose | 1.1 | 1.49 | 2.16 | 0.0158 | 0.0705 | 0.0009 | 0.0016 | 0.0654 | 0.0601 |
| α-ketoglutarate | 0.52 | 0.86 | 1.64 | 0.001 | 0.0244 | 0.0013 | 0.0022 | 0.2712 | 0.1627 |

Unknowns

A variety of unknowns were also observed which showed a relationship between: metabolite identity and metabolite level for healthy oral status, gingivitis and periodontal disease as shown in Table 5.

TABLE 5

| | H vs. G | | H vs. P | | | | | | |
|---|---|---|---|---|---|---|---|---|---|
| | p | q | p | | G vs. P | | MEAN | | |
| COMPOUND | value | value | value | q | P | Q | H | G | P |
| Dental 10461 | 0.00561 | 0.0549 | 2.00E−04 | 7.00E−04 | 0.0365 | 0.0456 | 0.89 | 1.25 | 1.96 |
| Dental 3138 | 0.0161 | 0.0705 | 0 | 2.00E−04 | 0.0021 | 0.0068 | 0.31 | 0.46 | 1.91 |
| Dental 2038 | 0.0389 | 0.1218 | 5.00E−04 | 0.004 | 0.0082 | 0.0151 | 1.54 | 1.1 | 0.72 |
| Dental 10864 | 0.7207 | 0.4203 | 0.0051 | 0.0058 | 2.00E−04 | 0.0021 | 1.37 | 1.09 | 0.56 |

What is claimed:

1. A method for diagnosing oral health in a subject comprising:
a. collecting a gingival crevicular fluid sample from the subject;
b. detecting a level of one or more metabolites in the gingival crevicular fluid sample; and
c. diagnosing the subject a having periodontal disease or oral status based on the level of the detected metabolite,
wherein the detected metabolite is at least one compound chosen from: a compound generated by amino acid metabolism, a compound generated in urea cycle; a compound generated in glutathion conversion; a compound generated in lipid metabolism; a compound generated in carbohydrate metabolism; a compound generated by nucleic acid metabolism; vitamins; and co-factors.

2. A method for diagnosing an oral health status in a subject comprising:
a. collected a gingival crevicular fluid from the subject;
b. detecting a level of metabolite in the gingival crevicular fluid sample;
c. comparing the level of detected metabolite in the gingival crevicular fluid sample to a metabolite reference level to thereby generate a differential level, wherein the metabolite reference level corresponds to one or more of the following: periodontal reference level or healthy reference level.
wherein the detected metabolite is at least one compound chosen from: a compound generated by amino acid metabolism, a compound generated in urea cycle; a compound generated in glutathion conversion; a compound generated in lipid metabolism; a compound generated in carbohydrate metabolism; a compound generated by nucleic acid metabolism; vitamins; and co-factors; and
d. based on the comparing, providing a diagnoses of the subject's oral health status.

3. The method of claim 2, wherein the differential level of the detected metabolite and the periodontal reference level correlates with periodontal disease.

4. The method of claim 2, wherein the differential level of the detected metabolite and the healthy reference level correlates with healthy oral status.

5. A method for monitoring periodontal disease in a subject comprising:
a. detecting a first level of at least one metabolite in a first gingiva crevicular fluid sample collected from the subject at a first point in time;
b. detecting second level of at least one metabolite in a second gingival crevicular fluid sample collected from the subject at a second point in time;
c. comparing the first detected metabolite level to the second detected metabolite level,
wherein a differential level of the second detected metabolite level relative to the first detected metabolite level indicates a change in periodontal disease of the subject.

6. The method of claim 5, wherein the differential level corresponds to a decrease in the second detected metabolite level relative to the first detected metabolite level indicating a decrease in the periodontal disease of the subject, and wherein the at least one metabolite is at least one member chosen from: inosine, hypoxanthine, xanthine, guanosine, guanine, leucine, isoleucine, lysie, methionine, phenyllalanine, proline, serine, threonine, tryptophan, tyrosine, valine, phenylacetic acid, α-hydroxyioscaproic acid, 5-amino valeric acid, choline, glycreol-3-phosphate, and N-acetylneuraminic acid.

7. The method of claim 5, wherein the differential level corresponds an increase in the second detected metabolite level relative to the first detected metabolite level indicating a decrease in periodontal disease of the subject, and wherein the at least one metabolite is at least one member chose from: uric acid, reduced glutathione, oxidized glutathion, ascorbic acid, and glutamine.

8. A method of determining an efficiency of a test compound useful in treating periodontal development in a mammal comprising the steps of:
detecting a post-treatment metabolite level from a gingival crevicular fluid sample collected from a subject after treatment with a test compound;
comparing the post-treatment metabolite level to one or more of the following: pre treatment metabolite levels of the subject, periodontal reference levels and healthy reference level; and
determining the efficiency of the test compound based on the comparison.

9. The method of claim 8, wherein the pre-treatment metabolite level may be obtained by steps comprising:
detecting a pretreatment metabolite level of a first gingival crevicular fluid sample collected from the subject at a first point in time; and
applying a dentifrice containing the test compound to an oral cavity of the subject according to a prescribed protocol;
wherein the post-treatment metabolite level is detected at a second point in time.

10. The method of claim 8, further comprising:
determining whether the test compound down-regulates at least one member chose from: inosine, hypoxanthine, xanthine, guanosine, guanine, leucine, isoleucine, lysie, methionine, phenyllalanine, proline, serine, threonine, tryptophan, tyrosine, valine, phenylacetic acid, α-hydroxyioscaproic acid, 5-amino valeric acid, choline, glycreol-3-phosphate, and N-acetylneuraminic acid.

11. The method of claim 9, further comprising:
determining whether the test compound up-regulates at least one member chose from: uric acid, reduced glutathione, oxidized glutathion, ascorbic acid, and glutamine.

12. A method of identifying a test compound useful in treating periodontal disease in a mammal, the method comprising contacting a cell with the test compound and determining whether the test compound down-regulates at least one member chose from: inosine, hypoxanthine, xanthine, guanosine, guanine, leucine, isoleucine, lysie, methionine, phenyllalanine, proline, serine, threonine, tryptophan, tyrosine, valine, phenylacetic acid, α-hydroxyioscaproic acid, 5-amino valeric acid, choline, glycreol-3-phosphate, and N-acetylneuraminic acid.

13. A method of claim 12, further comprising:
determining whether the test compound up-regulates at least one member chose from: uric acid, reduced glutathione, oxidized glutathion, ascorbic acid, and glutamine.

14. A method of identifying a test subject as a responder or non-responder to therapeutic agents while using a standard care protocol comprising the steps of:
generating a metabolite profile of a gingival crevicular fluid sample collected from the test subject, wherein the metabolite profile includes the metabolite identity and metabolite level;
comparing the metabolite profile of the test subject to a reference responder metabolite profile and a reference non-responder metabolite profile,
wherein the reference responder metabolite profile is generated from a reference responder subject which showed regression of periodontal disease while using a dentifrice containing a standard therapeutic agent according to the standard care protocol,
wherein the reference non-responder metabolite profile is generated from a reference non-responder subject which showed no change in periodontal disease while using a dentifrice containing a standard therapeutic agent according to the standard care protocol; and
based on the comparing, identifying the test subject as responder to therapeutic agents or non-responder to therapeutic agents.

15. The method of claim 14, wherein for the reference responder subject, the standard therapeutic agent down-regulates at least one member chose from: inosine, hypoxanthine, xanthine, guanosine, guanine, leucine, isoleucine, lysie, methionine, phenyllalanine, proline, serine, threonine, tryptophan, tyrosine, valine, phenylacetic acid, α-hydroxyioscaproic acid, 5-amino valeric acid, choline, glycreol-3-phosphate, and N-acetylneuraminic acid.

16. The method of claim 14, wherein for the reference responder subject, the standard therapeutic agent up-regulates at least one member chose from: uric acid, reduced glutathione, oxidized glutathion, ascorbic acid, and glutamine.

17. A method of identifying a test subject as a responder or non-responder to periodontal disease development while following a standard non-care protocol comprising the steps of:
generating a metabolite profile of the gingival crevicular fluid sample collected from the test subject, wherein the metabolite profile includes the metabolite identity and metabolite level;
comparing the metabolite profile of the test subject a reference responder metabolite profile and a reference non-responder metabolite profile,
wherein the reference responder metabolite profile is generated from a reference responder subject which developed periodontal disease while following the standard non-care protocol
wherein the reference non-responder metabolite profile is generated from a reference non-responder subject which failed to develop periodontal disease while following the standard non-care protocol; and
based on the comparing, identifying the test subject as responder to periodontal disease development or non-responder to periodontal disease development.

18. The method of claim 17, wherein for the reference responder subject, the standard non-care protocol up regulates at least one member chose from: inosine, hypoxanthine, xanthine, guanosine, guanine, leucine, isoleucine, lysie, methionine, phenyllalanine, proline, serine, threonine, tryptophan, tyrosine, valine, phenylacetic acid, α-hydroxyioscaproic acid, 5-amino valeric acid, choline, glycreol-3-phosphate, and N-acetylneuraminic acid.

19. The method of claim 17, wherein for the reference non-responder subject, the standard non-care protocol down-regulates at least one member chose from: uric acid, reduced glutathione, oxidized glutathion, ascorbic acid, and glutamine.

20. An oral care kit comprising:
one or more gingivitis crevicular fluid collection strips for collecting a gingival crevicular fluid sample and for recovery of metabolites contained in the gingival crevicular fluid sample;
a diagnosis of a subject's oral health based on the method of claim 5.

21. A dentifrice composition comprising:
an effective amount of an oral health metabolite therapeutic agent, wherein the therapeutic agent effects a change in metabolite levels over a time period of at least one month wherein the change metabolite level is at least 5 percent greater than a corresponding change in metabolite reference levels effected by a control dentifrice composition.

22. The dentifrice composition of claim 18, wherein the control dentifrice contains Triclosan.

23. A method to detect metabolites corresponding to periodontal disease in a subject's oral cavity comprising the steps of:
applying a dentifice containing a metabolite indicating composition to one or more sections of, test in the oral cavity, wherein said dentifrice contains a metabolite indicating composition which presents a user discernable indicator upon exposure to a metabolite and a metabolite level associated with periodontal disease.

24. A metabolite indicating dentifrice comprising:
a metabolite indicating composition which presents a user discernable indicator upon exposure to a metabolite and a metabolite level associated with periodontal disease.

25. The metabolite indicating dentifrice composition of claim 24, wherein the user discernable indicator corresponds to a change in color of the metabolite indicating dentifrice.

26. The metabolite indicating dentifrice composition of claim 24, wherein the metabolite is least one compound chosen from: a compound generated by amino acid metabolism, a compound generated in urea cycle; a compound generated in glutathion conversion; a compound generated in lipid metabolism; a compound generated in carbohydrate metabolism; a compound generated by nucleic acid metabolism; vitamins; and co-factors.

27. The metabolite indicating dentifrice composition of claim 24, wherein the metabolite level is indicative of up regulation of one or more of the following: inosine, hypoxanthine, xanthine, guanosine, guanine, leucine, isoleucine, lysie, methionine, phenyllalanine, proline, serine, threonine, tryptophan, tyrosine, valine, phenylacetic acid, α-hydroxyioscaproic acid, 5-amino valeric acid, choline, glycreol-3-phosphate, and N-acetylneuraminic acid.

28. The metabolite indicating dentifrice composition of claim 24 wherein the metabolite level is indicative of down regulation of one or more of the following: uric acid, reduced glutathione, oxidized glutathion, ascorbic acid, and glutamine.
